(19) United States
(12) Patent Application Publication
(10) Pub. No.: US 2026/0166237 A1
(43) Pub. Date: Jun. 18, 2026

(54) MULTIFUNCTIONAL THERAPY DEVICE WITH AN ATOMIZATION MODULE

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Mou Xiong, Shenzhen (CN)

(73) Assignee: SHENZHEN NUON MEDICAL EQUIPMENT CO., LTD., Shenzhen (CN)

(21) Appl. No.: 19/415,732

(22) Filed: Dec. 10, 2025

(30) Foreign Application Priority Data
Dec. 13, 2024 (CN) 2024230896224
May 29, 2025 (CN) 2025210918723

(51) Int. Cl. A61M 11/04; A61M 11/00; A61N 5/06

(52) U.S. Cl. CPC A61M 11/042; A61M 11/005; A61N 5/06; A61M 2205/051; A61M 2205/586; A61N 2005/0644

(57) ABSTRACT

Embodiments of the present invention disclose a multifunctional therapy device that integrates a mist-generation module with one or more additional treatment modalities to enhance therapeutic efficiency. The device includes a reservoir, a mist-producing unit, and a control module configured to regulate mist output parameters. The mist therapy hydrates and conditions the target area, thereby improving comfort and facilitating better penetration and responsiveness to complementary therapies such as phototherapy, microcurrent stimulation, thermal therapy, or vibration therapy. The system may further provide user-selectable operating modes and safety features. By coordinating mist delivery with other therapeutic functions, the device enhances overall treatment performance and broadens applicability in cosmetic, dermatological, and wellness care.

### TECHNICAL FIELD

The present invention relates to the field of personal care and skin-treatment devices, and more particularly to a multifunctional therapy device equipped with an atomization module. The invention encompasses beauty and therapeutic apparatuses capable of delivering liquid atomization in combination with one or more treatment modalities such as phototherapy, heating, microcurrent stimulation, vibration massage, and airflow-assisted mist delivery.

### BACKGROUND ART

Conventional phototherapy, thermal therapy, and vibration-based skin treatments often expose the skin to conditions that may cause dryness, irritation, or uneven therapeutic effects. For example, prolonged light exposure may increase transepidermal water loss, while heating or cooling elements can draw moisture away from the skin surface, reducing treatment comfort and efficacy. Mist-based hydration, when used in isolation, provides moisture but cannot deliver targeted therapeutic effects such as controlled heating, cooling, or photobiological action. In recent years, it has been recognized that integrating atomized mist delivery with other treatment modalities can significantly enhance overall performance

Moisture in the form of fine atomized particles helps maintain skin hydration during phototherapy or temperature-controlled treatment, thereby minimizing dryness and improving tolerance of the therapy. The presence of a water mist can also improve thermal conduction, enabling more uniform temperature transfer to the skin. Furthermore, hydrated skin may respond better to light-based and heat-based treatments, potentially improving treatment effectiveness, user comfort, and post-treatment recovery.

Many existing devices that employ atomization or mist-generation technology similarly exhibit limited functionality. Traditional atomizers are usually standalone units designed solely to release moisture or skincare formulations in the form of mist. These devices cannot often coordinate the atomization process with other treatment modalities such as vibration, heat, or phototherapy. As a result, users must manually combine multiple devices to achieve a multi-effect treatment, which can be inefficient and difficult to control in a synchronized manner.

Furthermore, current atomization systems frequently rely on basic structural arrangements that do not effectively manage airflow, mist dispersion, or interaction between atomized particles and other functional outputs. For example, the mist may not be uniformly distributed when combined with light or heat, or the atomization process may interfere with electrical or thermal components due to inadequate separation or protective structures. Such limitations restrict the adoption of atomizing elements in multifunctional therapeutic devices.

Another challenge observed in existing multi-module devices is the lack of structural flexibility. Devices equipped with multiple treatment modules often use rigid layouts that are not scalable or adaptable to different form factors. This makes it difficult to provide alternative embodiments of the same device concept using different body structures while maintaining consistent integration of the atomization unit and other therapy modules.

Therefore, there exists a need for a multifunctional therapy device that integrates an atomization module within a compact, unified structure along with one or more therapeutic modules, such as phototherapy components, heating modules, vibration elements, or microcurrent systems. Such a device should enable synchronized operation of atomization with other therapeutic functions, ensure efficient mist dispersion, and allow for different structural embodiments while preserving the core functional integration. The device should also improve convenience, versatility, and user experience compared to traditional single-function products.

### OBJECTS OF THE INVENTION

Some of the objects of the invention are as follows:

An object of the present invention is to provide a multifunctional therapy device incorporating an atomization module capable of delivering a fine mist or atomized liquid in a controlled manner.

Another object of the invention is to provide a multifunctional therapy device in which the atomization module can be effectively integrated with one or more treatment functionalities, such as phototherapy, microcurrent stimulation, heating, vibration massage, and airflow-assisted delivery, thereby enabling coordinated or independent operation of multiple therapeutic modes.

A further object of the invention is to offer a device structure that supports multiple alternative embodiments, allowing the atomization module and therapy components to be arranged in different configurations without compromising functional integration or performance consistency.

Another object of the invention is to improve the efficiency and uniformity of mist dispersion by providing optimized airflow pathways, nozzle arrangements, or coupling structures that facilitate smooth interaction between atomized particles and the selected therapy modules.

Another object of the invention is to enhance the convenience and usability of multifunctional treatment devices by providing a compact, ergonomically designed form factor that accommodates various modules while maintaining user comfort and operational simplicity.

A still further object of the invention is to reduce the need for multiple separate devices by offering a unified solution that performs multiple skincare or therapeutic functions, thereby improving user experience, reducing cost, and simplifying maintenance.

Yet another object of the invention is to provide a versatile platform capable of working with different liquids, skincare formulations, or therapeutic treatments, ensuring broad applicability across cosmetic, wellness, and dermatological use cases.

### SUMMARY OF THE INVENTION

According to a first aspect of the invention, a multifunctional therapy device is provided. The multifunctional therapy device comprising: a housing having an atomized mist outlet and a mounting part; a detachable atomizing module mounted on the mounting part, the atomizing module comprising a liquid storage chamber, an atomizing chamber, and an atomizing core; a phototherapy component disposed within the housing and corresponding to a light-transmitting surface surrounding the atomized mist outlet; a heat-conducting surface surrounding the atomized mist outlet; and a heating element disposed within the heat-conducting surface.

In one embodiment of the invention, the housing forms a Y-shaped configuration to facilitate user handling and comprising: a grip portion, a first extension portion having the atomized mist outlet, and a second extension portion having the mounting part.

In one embodiment of the invention, the heat-conducting surface and light-transmitting surface are concentric around the atomized mist outlet.

In one embodiment of the invention, the atomizing module comprises a first conductive element contacting a second conductive element in the housing to power the heating element.

In one embodiment of the invention, the atomizing module includes an atomizing chamber and a connecting channel allowing liquid from the liquid storage chamber to the atomizing core.

In one embodiment of the invention, the multifunctional therapy device further comprising: a main control board and a battery electrically connected to the atomizing module, phototherapy component, and heating element.

In one embodiment of the invention, the atomizing module is detachable for cleaning or replacement to reduce maintenance costs.

According to a second aspect of the present invention, a multifunctional therapy device is provided. The multifunctional therapy device comprises: a housing having a mounting part, a mist outlet channel, and an atomized mist outlet; an atomizing module detachably mounted on the mounting part, the atomizing module comprising a liquid storage chamber, an atomizing chamber, and an atomizing core configured to atomize a liquid from the liquid storage chamber into an atomized mist in atomizing chamber; a fan disposed within the housing, the fan is configured to direct the atomized mist toward the atomized mist outlet; and a main control board electrically connected to the atomizing module.

In one embodiment of the invention, the atomizing core is a ceramic core with a heating element.

In one embodiment of the invention, the atomizing core is an ultrasonic atomizing sheet.

In one embodiment of the invention, the mounting part includes a mounting groove with a first through hole and a second through hole aligned with an air inlet and an air outlet of the atomizing module.

In one embodiment of the invention, the multifunctional therapy device further comprising a sealing pad disposed on the mounting portion to prevent mist leakage.

In one embodiment of the invention, the multifunctional therapy device further comprising one or more magnetic attractors to secure the atomizing module to the housing.

In one embodiment of the invention, the atomizing module includes a liquid replenishment port with a detachable sealing cap.

According to a third aspect of the present invention, a multifunctional therapy device is provided. The multifunctional therapy device comprising: a housing; a liquid storage chamber comprising a first container and a second container, the first container being detachably connected to the housing through a mounting part having a communication port, and the second container having a second opening in fluid communication with the first opening of the first container through the communication port; the second container further comprising a first part and a second part arranged in an intersecting configuration, the second part including a water outlet and containing absorbent cotton, a portion of the absorbent cotton extending into the first part; an atomizing core arranged to receive liquid from the water outlet guided by the absorbent cotton and to atomize the liquid; and an atomized mist outlet configured to discharge atomized mist generated by the atomizing core, wherein the second part is inclined such that liquid guided by the absorbent cotton is delivered upward toward the atomizing core in alignment with the spray direction of the atomized mist.

In one embodiment of the invention, the first container comprises a mounting housing open at both ends and a sealing cap fitted onto an upper end of the mounting housing.

In one embodiment of the invention, the absorbent cotton is positioned so that one end extends into the first part to draw liquid by capillary action toward the atomizing core.

In one embodiment of the invention, the atomizing core includes a piezoelectric atomizing plate.

In one embodiment of the invention, the atomizing core comprises an atomizing plate disposed within an atomizing chamber located between the water outlet and the atomized mist outlet.

In one embodiment of the invention, the central axis of the atomized mist outlet is parallel to the central axis of the water outlet to reduce flow resistance and ensure smooth ejection of atomized mist.

In one embodiment of the invention, the second part gradually tilts away from the first container from bottom to top to facilitate upward liquid delivery through the absorbent cotton.

In one embodiment of the invention, the liquid storage chamber further comprises an adapter positioned between the first container and the second container, the adapter having two openings respectively connected to the communication port and the second opening and including sealing rings or sealing sleeves to prevent leakage.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “beauty device”, “therapy device”, or “physiotherapy device” refers to the device of the present invention configured to perform atomization, phototherapy, thermal therapy, or combined treatment.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, or combinations thereof.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the term “atomizing module,” “atomizer,” or “atomization unit” refers to the component configured to store liquid and generate atomized mist.

In the context of the specification, the term “liquid container,” “liquid storage chamber,” or “reservoir” refers to a chamber or container configured to hold liquid for atomization.

In the context of the specification, the term “heating element,” “temperature control element,” or “thermal element” refers to any suitable device or structure for heating or cooling the treatment surface or liquid.

In the context of the specification, the term “light-emitting element,” “phototherapy component,” or “light-transmitting surface” refers to a component configured to emit light for skin treatment.

In the context of the specification, the term “mounting part,” “mounting groove,” or “mounting housing” refers to a structure configured to hold, position, or support the atomizing module.

In the context of the specification, the term “sealing pad,” “retaining ring”, or “gasket” refers to a component configured to provide a sealed connection between structural elements, preventing liquid or mist leakage.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 shows a first configuration of a multi-functional therapy device, in accordance with an embodiment of the present invention.

FIG. 2 is the multi-functional therapy device of FIG. 1 with an atomizing module in a detached state, in accordance with an embodiment of the present invention.

FIG. 3 shows the atomizing module, in accordance with an embodiment of the present invention.

FIG. 4 shows a cross-sectional view of the multi-functional therapy device of FIG. 1, in accordance with an embodiment of the present invention.

FIG. 5 is an enlarged view of point A in FIG. 4, in accordance with an embodiment of the present invention.

FIG. 6 is an enlarged view of point B in FIG. 4, in accordance with an embodiment of the present invention.

FIG. 7 is another cross-sectional view of the multi-functional therapy device in FIG. 1, in accordance with an embodiment of the present invention.

FIG. 8 is an enlarged view of point C in FIG. 7, in accordance with an embodiment of the present invention.

FIG. 9 shows a second configuration of a multi-functional therapy device, in accordance with an embodiment of the present invention.

FIG. 10 is a front view of the multi-functional therapy device in FIG. 9, in accordance with an embodiment of the present invention.

FIG. 11 is a top view of the multi-functional therapy device in FIG. 9, in accordance with an embodiment of the present invention.

FIG. 12 is a cross-sectional view along section AA in FIG. 11, in accordance with an embodiment of the present invention.

FIG. 13 is an enlarged view of point B in FIG. 12, in accordance with an embodiment of the present invention.

FIG. 14 is an exploded view of the multifunctional therapy device in FIG. 9, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a multifunctional therapy device incorporating an atomization module configured to deliver a fine mist or atomized liquid for skincare or therapeutic purposes. The multifunctional therapy device includes a housing that supports and protects various functional modules and internal components. The housing is either formed as a handheld body, a mask-like structure, a surface-contact device, or another ergonomic configuration suitable for directing treatment toward a user's skin.

A reservoir is disposed within the housing for storing a liquid to be atomized, such as water, toner, serum, or other skincare compositions. The reservoir is either detachable or integrated, and includes sealing elements or flow-control structures to prevent leakage during use.

An atomization module is fluidly coupled to the reservoir. The atomization module further includes an ultrasonic vibrating element, a mesh atomizer, a heating element, or other mechanism capable of generating fine mist particles. The module typically comprises an atomization chamber, a liquid-feeding structure, and an atomization outlet through which the mist is discharged. The atomization module is configured to be mounted directly within the housing or supported by a dedicated frame to ensure stable operation and efficient mist ejection. A main control board is electrically connected to the atomization module and other functional modules. The control board manages operational modes, timing, power supply, safety protection, and coordinated functioning of multiple therapies. The control board may include a microcontroller, power conversion elements, driver circuits, sensors, and wireless communication components.

In an embodiment, the multifunctional therapy device further includes one or more stimulation elements integrated alongside the atomization module. These may include: a phototherapy element, a microcurrent element, a heating element, a cooling element, a piezoelectric element, etc., or combinations thereof, without limitation. These stimulation elements can be integrated within or disposed on the stimulation unit to provide multiple therapeutic effects during user operation.

In an embodiment, the stimulation element can be a phototherapy element. The phototherapy element is configured to emit light of specific wavelengths, such as red, blue, or near-infrared light, to promote skin rejuvenation, enhance microcirculation, or assist in the absorption of cosmetic solutions. The light emitted from the phototherapy element can penetrate the skin to stimulate cellular activity, support collagen production, and improve overall skin texture and tone.

In an embodiment, the stimulation element can be a microcurrent element. The microcurrent element generates low-intensity electrical currents that mimic the body's natural bioelectric signals. Application of microcurrents via the stimulation unit can stimulate facial or scalp muscles, enhance cellular metabolism, and promote absorption of topical solutions. Microcurrent therapy also improves skin firmness and elasticity over time.

In an embodiment, the stimulation element can be a thermal element. The thermal element provides localized warmth to the stimulation unit, which can improve blood circulation, relax tissues, and reduce muscle tension. Heat generated by the thermal element or transferred from the adjacent heating element can also enhance penetration and absorption of cosmetic solutions, providing a hot-compress effect for user comfort.

The thermal element also allows the stimulation unit to deliver a cold-compress effect, which can soothe irritated skin, reduce inflammation, and tighten pores. Cooling therapy can be particularly beneficial after phototherapy, microcurrent treatment, or application of active cosmetic formulations.

In an embodiment, the stimulation element can be a piezoelectric element. The piezoelectric element converts electrical signals into mechanical vibrations, allowing the stimulation unit to generate precise vibrational massage. These vibrations help in spreading cosmetic solutions evenly across the skin, improve local microcirculation, and provide gentle stimulation to the underlying tissues. Piezoelectric stimulation can be combined with other elements, such as heating or phototherapy, to achieve a synergistic therapeutic effect.

In an embodiment, the stimulation element can be a vibration module that produces mechanical stimulation for massaging, soothing, or enhancing mist penetration.

In an embodiment, the stimulation element can be an airflow generation module, such as a micro-fan, for assisting in guiding or dispersing atomized particles across the target surface.

The housing further includes user-operable controls, such as buttons, touch interfaces, or switches for selecting operational modes, adjusting intensity, or activating specific therapies. A display element or indicator light can present status information, including battery level, mode selection, or device readiness.

Airflow channels are formed within the housing to direct ambient or forced air toward the atomization outlet to improve mist distribution. These channels include: an inlet opening, air-guiding ducts, or outlet passages that work in coordination with the airflow generation module.

The multi-functional therapy device also includes a removable or openable cover configured to provide access to the reservoir, atomization chamber, or other internal components for refilling, cleaning, or replacing consumables. In certain implementations, the device further incorporates replaceable elements such as filter media, mesh atomizers, or pre-filled liquid cartridges.

Power is supplied from an internal rechargeable battery or an external power source. Charging may be achieved through a USB port, magnetic charging interface, or docking system. The control board may include battery management circuitry to ensure safe charging and optimized power distribution.

The multifunctional configuration of the therapy device allows the atomization module to operate independently or in combination with one or more stimulation elements. For example, phototherapy may be activated simultaneously with atomization to enhance treatment effectiveness. Heating or vibration may be synchronized with mist delivery for deeper penetration or improved comfort. The device may include preset or user-selectable programs to operate multiple modules in a sequence or simultaneously.

In an embodiment, the multi-functional therapy device further comprises a dual-liquid atomizing module, wherein the atomizing module includes a first liquid storage chamber and a second liquid storage chamber, each independently connected to an atomizing core. The first and second atomizing cores can operate simultaneously or sequentially to atomize liquids of different compositions, such as water and a nutrient solution, into the atomizing chamber. This allows the device to deliver a combined or customizable treatment to the skin. A user-selectable switch or control interface can be provided to select the atomization sequence or combination ratio of the liquids.

In some embodiments, the atomizing module is configured to provide adjustable atomization intensity. The atomization output, including mist particle size and flow rate, can be controlled by varying the operating frequency of the atomizing core or adjusting the speed of the fan disposed within the first housing. This enables the user to select a gentle, fine mist for sensitive skin or a more intense spray for rapid hydration. The main control board can automatically adjust the atomization intensity based on preset modes.

In some embodiments, the multifunctional therapy device further includes at least one sensor configured to monitor skin or environmental conditions. The sensor includes, but is not limited to, a temperature sensor, a humidity sensor, or a liquid level sensor disposed in the liquid storage chamber. The main control board receives data from the sensors and automatically adjusts the atomization operation, fan speed, phototherapy intensity, or heating/cooling of the treatment surface, thereby optimizing treatment efficiency and safety. An alert can be provided to the user if the liquid level is low or if abnormal conditions are detected.

In some embodiments, the phototherapy component comprises multiple light-emitting elements capable of emitting different wavelengths, such as red, blue, green, or infrared light. The user can select a specific wavelength or combination of wavelengths for targeted skin treatment, including hydration, anti-acne, or anti-aging effects. The light-emitting elements can be arranged around the atomizing outlet, allowing atomized mist to simultaneously contact the treated skin. The main control board can provide automated cycling between different wavelengths during the treatment session.

In some embodiments, the multifunctional therapy device is configured as a compact, portable, or wearable device. The housing is reduced in size while maintaining the atomizing module, phototherapy component, and heat/cold elements. The device can be powered by a rechargeable battery or replaceable battery cartridge, and may include a detachable liquid container for ease of use and refill. The device can include a clip or strap for wearable use, allowing continuous or hands-free operation.

In some embodiments, the device includes an airflow-guiding system that directs atomized mist evenly across the treatment surface. The airflow system can comprise a fan and a set of ducts surrounding the atomizing outlet. This configuration ensures uniform distribution of mist onto the skin and improves the penetration and coverage of hydration. Optionally, the airflow can be adjusted in direction or intensity based on user preference.

The invention is not limited to any single structural layout. The atomization module, therapy modules, and airflow pathways may be arranged in various configurations depending on the intended form factor. For instance, in one embodiment, the atomization module may be centrally positioned within the device, while in another embodiment, it may be located near an edge or distributed across multiple outlets. Similarly, the therapeutic components may be arranged around the atomization outlet, adjacent to it, or in separate regions of the device body.

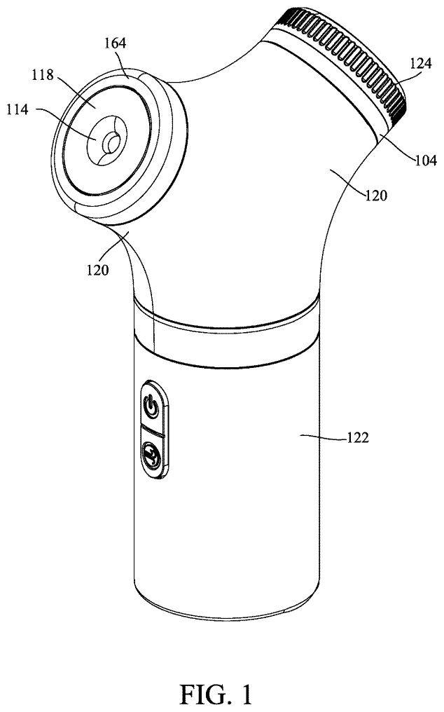

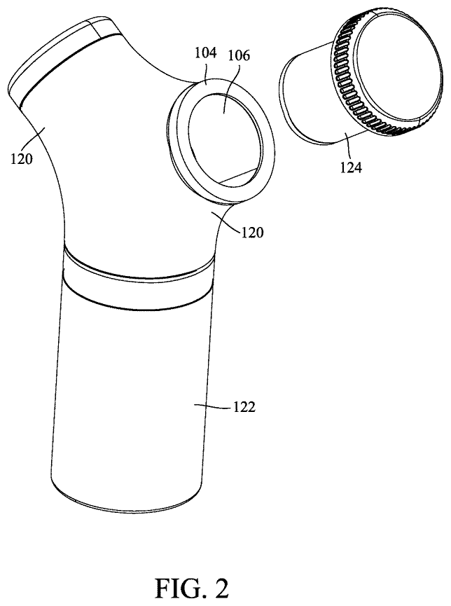

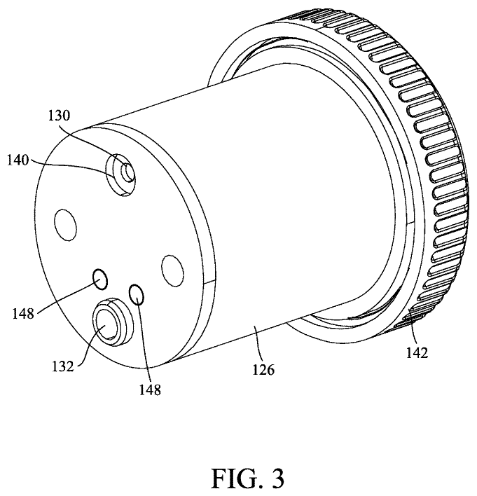

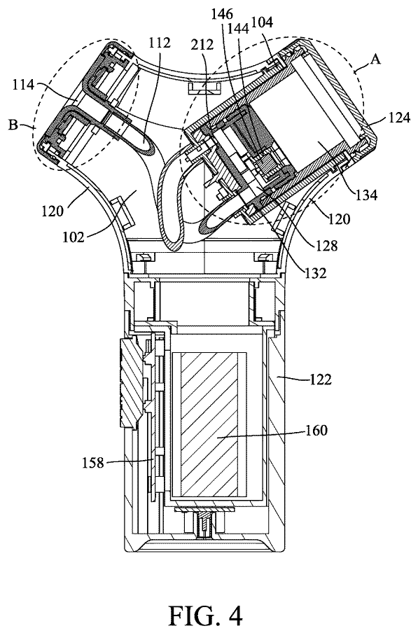

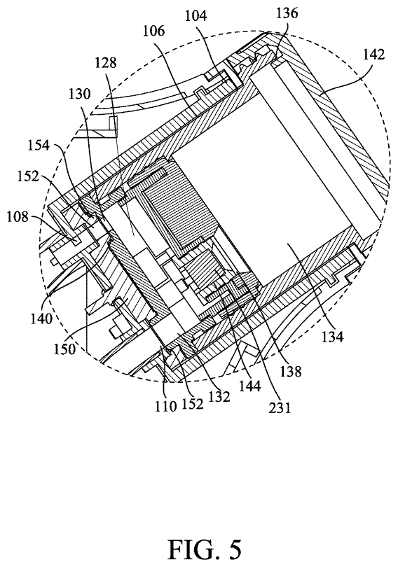

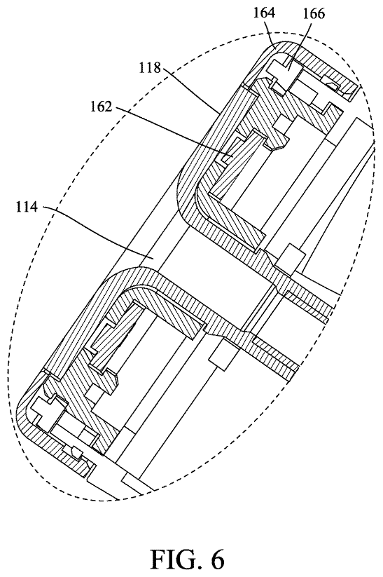

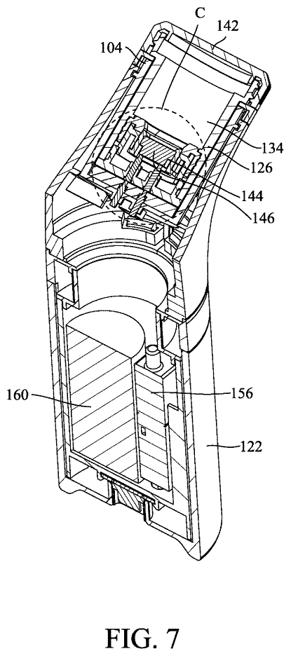

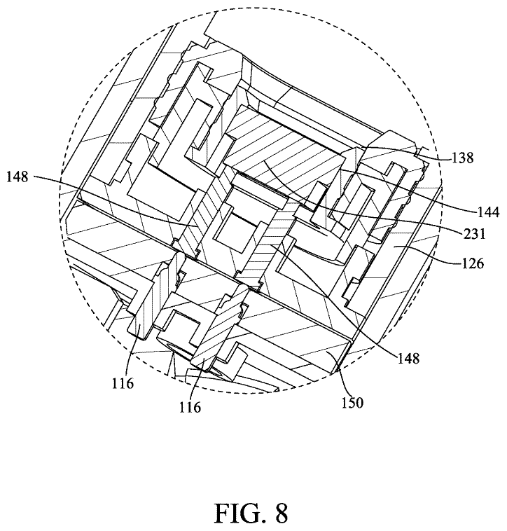

FIGS. 1 to 8 show a first configuration of the multi-functional therapy device. Referring to FIGS. 1 to 8, the multi-functional therapy device includes a housing 102 and a detachable atomizing module 124. The housing 102 is provided with a mounting part 104, a mist outlet channel 112, and an atomized mist outlet 114. One end of the mist outlet channel 112 communicates with the atomized mist outlet 114, while the other end extends toward the mounting part 104. The mounting part 104 is arranged at a position spaced apart from the atomized mist outlet 114. The atomizing module 124 is detachably mounted on the mounting part 104 and includes an atomizing chamber 128 and a liquid storage chamber 134. When the atomizing module 124 is installed, the atomizing chamber 128 is brought into communication with the mist outlet channel 112.

The atomizing chamber 128 and the liquid storage chamber 134 are interconnected by a connecting channel 138. The atomizing module 124 further includes an atomizing core 144, which is located within the atomizing chamber 128 or within the connecting channel 138. Liquid stored in the liquid storage chamber 134 is guided toward the atomizing core 144, where it is atomized and delivered into the atomizing chamber 128. The resulting water mist is then expelled through the mist outlet channel 112 and the atomized mist outlet 114 to provide skin hydration.

The atomizing module 124 is detachable and is easily removed and cleaned after use. Moreover, in the event of a malfunction, the atomizing module 124 can be directly replaced without requiring disassembly of the entire device, thereby simplifying operation. Compared with the need to purchase an entirely new therapy device, this detachable structure reduces maintenance costs and improves user convenience.

In some embodiments, the atomizing chamber 128 is provided with an air inlet 130 and an air outlet 132, wherein the air outlet 132 communicates with the mist outlet channel 112. The multi-functional therapy device further includes a fan 156 disposed inside the housing 102, the air outlet of which is connected to the air inlet 130. The fan 156 blows the mist generated within the atomizing chamber 128 toward the atomized mist outlet 114, thereby increasing the mist discharge speed and reducing the likelihood of mist accumulation or droplet formation due to prolonged residence time. This configuration effectively enhances the atomization performance. The air outlet of the fan and the air inlet 130 are configured to connect through a flexible hose. In an alternate embodiment, the atomizing chamber 128 is configured as an open structure and together with the mounting part 104 forms a relatively closed chamber.

In some embodiments, the mounting part 104 includes a mounting groove 106. The bottom wall of the mounting groove 106 is provided with a first through hole 108 and a second through hole 110. The first through hole 108 communicates with the air outlet and is positioned corresponding to the air inlet 130, while the second through hole 110 communicates with the mist outlet channel 112 and is positioned corresponding to the air outlet 132. The atomizing module 124 is inserted into and detachably received within the mounting groove 106. When inserted, the first through hole 108 aligns with the air inlet 130, and the second through hole 110 aligns with the air outlet 132. This arrangement ensures stable positioning of the atomizing module 124 and avoids sliding friction between the module and the openings of the through holes during disassembly or assembly, thereby reducing wear, improving sealing performance, and prolonging service life. In other embodiments, the mounting part 104 may alternatively be provided with an external thread, and the atomizing module 124 may be secured by threaded engagement.

In some embodiments, the atomizing core 144 is a ceramic atomizing core having a heating element 146 disposed on the side of the atomizing core 144 facing the atomizing chamber 128. The atomizing core 144 includes a ceramic body with a porous structure and the heating element 146 mounted thereon. The porous ceramic body allows the liquid stored in the liquid storage chamber 134 to permeate into the ceramic body. When the heating element 146 is energized, the liquid absorbed within the ceramic body is rapidly heated and atomized. Because the heating element 146 is positioned adjacent to the atomizing chamber 128, the generated mist is efficiently delivered into the atomizing chamber 128, thereby improving atomization performance. In alternative embodiments, the atomizing core 144 may be implemented as an ultrasonic atomizing sheet.

In some embodiments, the atomizing module 124 further includes two first conductive elements 148, with the heating element 146 electrically connected between the two first conductive elements 148. The bottom wall of the mounting groove 106 is provided with two second conductive elements 116, and each of the first conductive elements 148 corresponds to and abuts one of the second conductive elements 116. The multifunctional therapy device also includes a main control board 158 disposed within the housing 102 and electrically connected to the second conductive elements 116. Specifically, the two first conductive elements 148 make contact with respective ends of the heating element 146, and the opposite ends of the first conductive elements 148 extend outward from the atomizing module 124 so that, upon insertion of the atomizing module 124 into the mounting groove 106, the first conductive elements 148 directly abut the second conductive elements 116. This structure minimizes sliding friction during installation, reduces wear, and ensures stable electrical conductivity. In some implementations, the second conductive elements 116 may be spring-loaded pins to maintain reliable electrical contact. Alternatively, the first conductive elements 148 may be spring pins.

In some embodiments, the bottom wall of the mounting groove 106 is equipped with a first magnetic attractor, and the atomizing module 124 is provided with a corresponding second magnetic attractor. At least one of the first and second magnetic attractors may be a permanent magnet, and one or more first magnetic attractors may be used. When the atomizing module 124 is inserted into the mounting groove 106, the first and second magnetic attractors magnetically engage with each other, thereby enhancing the installation stability of the atomizing module 124. In addition, the arrangement of the first conductive elements 148 and second conductive elements 116 ensures secure contact and reliable electrical conduction.

In some embodiments, the multi-functional therapy device further includes a sealing pad 150 disposed on the bottom wall of the mounting groove 106. The sealing pad 150 is provided with clearance through-holes 152 corresponding to the positions of the first through hole 108 and second through hole 110. When the atomizing module 124 is inserted into the mounting groove 106, the atomizing module 124 presses against the sealing pad 150, and the sealing pad 150 seals the gaps between the first through hole 108 and the air inlet 130, and between the second through hole 110 and the air outlet 132, thereby reducing leakage and improving atomization efficiency.

In some embodiments, one of the sealing pad 150 and the atomizing module 124 is provided with a positioning rib 154, while the other is provided with a positioning groove 140 that engages with the positioning rib 154. This arrangement facilitates the accurate positioning and secure installation of the atomizing module 124, thereby preventing displacement of the air inlet 130 and the air outlet 132. Optionally, one of the air inlets 130 and the air outlet 132 may be provided with the positioning groove 140, while the positioning rib 154 is disposed around the edge of the corresponding clearance through-hole 152.

In some embodiments, the atomizing module 124 includes a mounting housing 126 and a sealing cap 142. The mounting housing 126 defines the atomizing chamber 128, the liquid storage chamber 134, and a liquid replenishment port 136 in communication with the liquid storage chamber 134. The liquid replenishment port 136 is located on the side of the liquid storage chamber 134 opposite the atomizing chamber 128. The sealing cap 142 is detachably mounted at the liquid replenishment port 136 and is positioned outside the mounting part 104. This configuration allows the user to replenish liquid into the liquid storage chamber 134 by simply opening the sealing cap 142, even when the atomizing module 124 is installed within the mounting groove 106, thereby improving convenience.

In some embodiments, the housing 102 is further provided with a light-transmitting surface 118 surrounding the atomized mist outlet 114. A multiple light-emitting elements 162 is disposed within the housing 102 and corresponds to the light-transmitting surface 118. During use, the multiple light-emitting elements 162 emit therapeutic light to the user's skin. By arranging the light-transmitting surface 118 around the atomized mist outlet 114, atomization may be activated concurrently with phototherapy, enabling the atomized mist to contact the skin being irradiated. This helps hydrate the skin and enhances the overall treatment effect.

In some embodiments, the multifunctional therapy device further includes a heat-conducting surface 164 and a temperature control element 166. The heat-conducting surface 164 is mounted on the housing 102 and encircles the atomized mist outlet 114, while the temperature control element 166 is positioned within the heat-conducting surface 164. In operation, the temperature control element 166 generates heat and transfers it to the heat-conducting surface 164, enabling thermal treatment of the skin. By placing the heat-conducting surface 164 around the atomized mist outlet 114, atomization can be performed simultaneously with heat application, providing moisture during heating and improving skin condition. The temperature control element 166 may be implemented as a heating wire, heating film, or other suitable structure. When both the multiple light-emitting elements 162 and the heat-conducting surface 164 are provided, the heat-conducting surface 164 may surround the light-transmitting surface 118; alternatively, the light-transmitting surface 118 may be arranged around the heat-conducting surface 164.

In some embodiments, the multifunctional therapy device further includes a main control board 158 and a battery 160 housed within the housing 102. The battery 160 and the atomizing module 124 are electrically connected to the main control board 158, allowing the main control board 158 and the atomizing module 124 to be powered by the battery 160. This reduces reliance on external power cables and improves portability. Optionally, the multifunctional therapy device may also include a charging interface electrically connected to the main control board 158 for recharging the battery 160 from an external power source. In other embodiments, the battery 160 may be configured as a removable battery.

In some embodiments, the housing 102 includes a grip portion 122 and two extension portions 120. The two extension portions 120 extend from one end of the grip portion 122 in opposite directions. One extension portion 120 is provided with the atomized mist outlet 114, while the other extension portion 120 carries the mounting part 104. Thus, the grip portion 122 and the two extension portions 120 form a generally Y-shaped structure, which provides a comfortable grip and reduces interference between the atomizing module and the user's skin during operation, thereby enhancing ease of use.

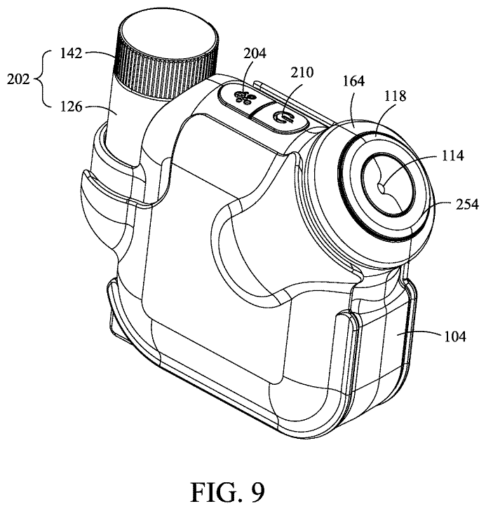

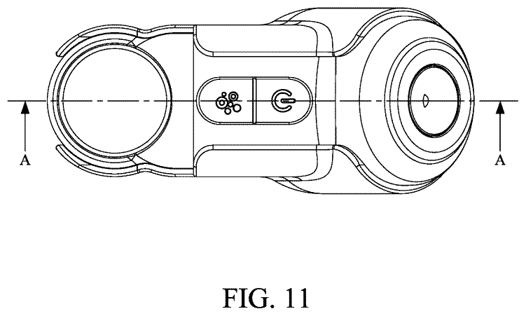

FIG. 9 to 14 illustrates another configuration of the multi-functional therapy device showing the positioning of the atomization module. FIG. 9 to 14 shows a second embodiment of the multifunctional therapy device. This embodiment shares the same inventive concept as FIGS. 1 to 8, with the atomization module as the core functional component. Only the structural arrangement of the housing, mounting, and associated components differs.

Referring to FIGS. 9 to 14, the multifunctional therapy device includes a main body 200, a liquid storage chamber 134, an atomizing core 144, a light-emitting element 162, and a temperature control element 166. The main body 200 has a treatment surface 254 and an atomized mist outlet 114 disposed on the treatment surface 254. The treatment surface 254 includes a light-transmitting surface 118 and a heat-conducting surface 164, both surrounding the atomized mist outlet 114. The liquid storage chamber 134 is fixed to the main body 200. The atomizing core 144 is installed on the housing 102 and is configured to atomize the liquid from the liquid storage chamber 134, directing the atomized liquid out through the atomized mist outlet 114. The light-emitting element 162 corresponds to the light-transmitting surface 118, while the temperature control element 166 corresponds to the heat-conducting surface 164 to regulate its temperature.

In this embodiment, the multifunctional therapy device integrates phototherapy, thermal (hot/cold) therapy, and hydration functions. During phototherapy or hot/cold compress treatment, the atomizing core 144 atomizes the liquid in the liquid storage chamber 134 and emits a fine water mist to hydrate the skin. Furthermore, since the light-emitting element 162 and the temperature control element 166 are arranged around the atomized mist outlet 114, the atomized water vapor envelops the skin being treated, preventing dryness caused by light exposure or thermal therapy. This design achieves a synergistic effect, providing a moisturizing benefit that enhances the overall efficacy of the treatment.

The temperature control element 166 may be a heating or cooling component, such as a semiconductor, heating wire, or heating plate. It generates heat or cold and transfers it to the heat-conducting surface 164 to adjust its temperature, thereby allowing the heat-conducting surface 164 to apply controlled heat or cold to the skin. The heat-conducting surface 164 is formed from a material with high thermal conductivity, enabling rapid and uniform temperature transfer.

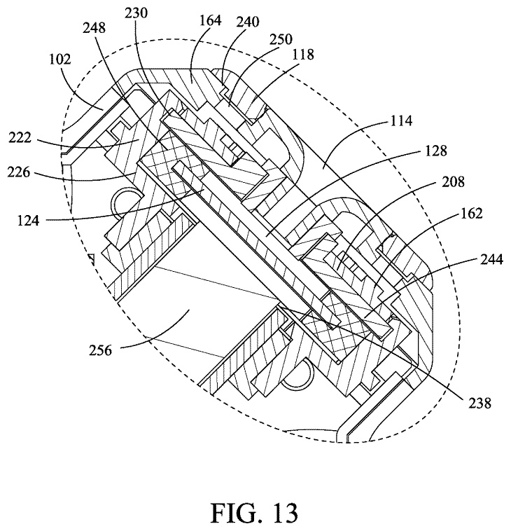

Referring to FIG. 13, in some embodiments, the liquid storage chamber 134 is provided with a water outlet 238, and an atomizing chamber 128 is arranged between the atomized mist outlet 114 and the water outlet 238. The atomizing core 144 may be implemented as an atomizing plate located within the atomizing chamber 128. The water outlet 238 is equipped with absorbent cotton 256, which guides liquid to the atomizing plate via capillary action. When powered, the atomizing plate undergoes piezoelectric-induced high-frequency oscillations, breaking down water molecules and generating a naturally drifting water mist. This process produces a fine atomized spray for skin hydration.

In other embodiments, the atomizing core 144 may alternatively be a heating element, which can be disposed either inside or outside the liquid storage chamber 134. When the heating element heats the liquid within the liquid storage chamber 134, the liquid generates water vapor, which is then expelled through the atomized mist outlet 114.

Additionally, the multifunctional therapy device is equipped with brackets for securing the atomizing core 144, the light-emitting element 162, and the temperature control element 166. These components can either share a common bracket or be mounted on separate brackets, depending on the design configuration.

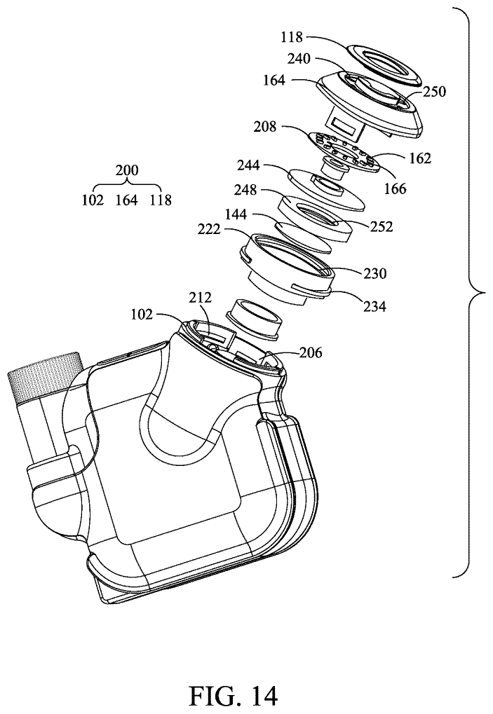

Referring to FIGS. 13 and 14, in some embodiments, the multifunctional therapy device further includes a first support 222, a second support 244, and a fixing ring 248. The first support 222 is annular and installed within the housing 102, sealingly fitted over the water outlet 238. The second support 244 is mounted on the first support 222, also annular, and sealingly fitted over the atomized mist outlet 114. The fixing ring 248 is positioned inside the first support 222, between the second support 244 and the water outlet 238, serving as a sealing element to ensure a secure connection between the first and second supports.

A circular groove 252 is formed on the inner circumferential surface of the fixing ring 248, into which the edge of the atomizing core 144 is embedded. The light-emitting element 162 and the temperature control element 166 are fixed to, and electrically connected with, the circuit board 208, which is mounted on the side of the second support 244 opposite to the fixing ring 248.

In this embodiment, at the lower end of the atomizing chamber 128, the first support 222 is sealed with the liquid storage chamber 134 to prevent water mist from escaping downward. At the upper end of the atomizing chamber 128, the second support 244 is sealed with the housing 102 to prevent water mist from escaping upward. In the middle portion of the atomization chamber 128, the fixing ring 248 seals the connection between the first support 222 and the second support 244, preventing water mist from escaping at their interface. This arrangement ensures a fully airtight connection between the atomized mist outlet 114 and the water outlet 238, so that all liquid or vapor from the water outlet 238 is effectively sprayed out through the atomized mist outlet 114.

Embedding the edge of the atomizing core 144 into the circular groove 252 of the fixing ring 248 not only secures the atomizing core 144 in place but also protects it from potential damage due to collisions with hard components.

Furthermore, the circuit board 208, the light-emitting element 162, and the temperature control element 166 are all positioned outside the atomizing chamber 128, preventing safety hazards and ensuring reliable electrical contact by avoiding exposure of these electronic components to water mist.

In some embodiments, the circuit board 208 is annular, and the first support 222 and the second support 244 together form an annular groove that houses the circuit board 208. This groove aids in precisely positioning the circuit board 208. Additionally, the circuit board 208 can be further secured either by bonding to the second support 244 or by fastening with screws.

The light-emitting element 162 and the temperature control element 166 are arranged around the atomized mist outlet 114. Specifically, multiple light-emitting elements 162 and a temperature control element 166 can be distributed at intervals along the circumference of the circuit board 208, with the temperature control element 166 positioned between every two adjacent light-emitting elements 162. In this embodiment, the temperature control element 166 is implemented as a heating resistor.

In some embodiments, the inner circumferential surface of the first support 222 is provided with an overlapping protrusion 226 and a mounting slot 230. The fixing ring 248 overlaps the side of the overlapping protrusion 226 facing the second support 244. The mounting slot 230 is annular, and the edge of the second support 244 is inserted into the mounting slot 230, thereby restricting the vertical movement of the second support 244. The second support 244 also contacts the fixing ring 248. In this embodiment, the fixing ring 248 is clamped between the second support 244 and the overlapping protrusion 226, preventing it from moving. Simultaneously, both the upper and lower ends of the atomizing core 144 are spaced apart from the overlapping protrusion 226 and the second support 244, protecting the atomizing core 144 from potential collisions.

Referring to FIG. 14, the outer peripheral surface of the first support 222 is provided with a limiting protrusion 234, and the inner wall of the housing 102 is provided with a corresponding limiting groove 212 for engagement with the limiting protrusion 234. Optionally, multiple limiting protrusions 234 can be arranged at intervals along the circumference of the first support 222, with corresponding multiple limiting grooves 212 provided in a one-to-one relationship. Alternatively, in other embodiments, the limiting groove 212 may be formed as a single closed annular structure, allowing the limiting protrusion 234 to engage at any position along the circumference. The limiting protrusion 234 may take various shapes, such as arc-shaped, hemispherical, or square.

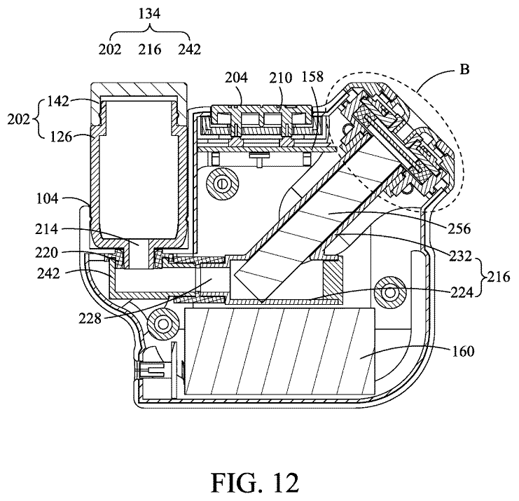

Referring to FIG. 12, in some embodiments, the liquid storage chamber 134 comprises a first container 202 and a second container 216. A mounting part 104 is formed on the outer surface of the housing 102, with a communication port 220 provided at the bottom of the mounting part 104. The first container 202 is detachably connected to the housing 102, with one end having a first opening 214 inserted into the mounting part 104. The second container 216 is located inside the housing 102 and has a second opening 228 and a water outlet 238. The second opening 228 communicates with the first opening 214 via the communication port 220. In this embodiment, the first container 202 can be removed from the housing 102 without disassembling the entire device, facilitating cleaning, replacement, or timely addition of liquid.

The first container 202 and the mounting part 104 are tightly fitted together. Alternatively, the first container 202 may include a locking protrusion on its outer periphery, which engages with a corresponding locking groove formed on the wall of the mounting part 104 to secure the container in place.

The first container 202 comprises a mounting housing 126 and a sealing cap 142. The mounting housing 126 is open at both ends, with the lower opening forming the first opening 214 that communicates with the communication port 220. The sealing cap 142 is fitted onto the upper end of the mounting housing 126, allowing liquid to be added when the sealing cap 142 is opened.

The second container 216 includes a first part 224 and a second part 232 arranged in an intersecting configuration. The first part 224 is arranged horizontally below the first container 202, with one end having a second opening 228. The second part 232 is positioned above the first part 224 and includes a water outlet 238. Absorbent cotton 256 is inserted into the second part 232, with one end extending into the first part 224. This configuration, compared to arrangements where both parts are strictly horizontal or vertical, helps prevent the device from becoming excessively large in a single direction.

Optionally, the second part 232 is inclined. Specifically, the second part 232 gradually tilts away from the first container 202 from bottom to top, with the central axis of the atomized mist outlet 114 parallel to the central axis of the water outlet 238. In this arrangement, the absorbent cotton 256 guides liquid upward toward the atomizing core 144, where it is atomized and sprayed at an angle through the water outlet 238. The guiding direction of the absorbent cotton aligns with the spray direction of the atomized mist, reducing flow resistance and ensuring smooth ejection of the water mist.

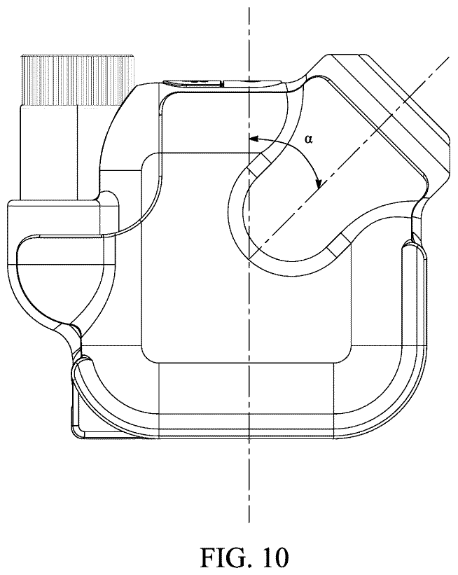

Referring to FIG. 10, optionally, the central axis of the atomized mist outlet 114 is set at an angle α relative to the vertical direction. When the multifunctional therapy device is placed on a table, the tilted atomized mist outlet 114 can spray water mist directly onto the user's face, providing an ergonomic design. The angle α can be, for example, 30°, 45°, or another suitable angle.

Referring to FIG. 12, the liquid storage chamber 134 further includes an adapter 242 positioned between the first container 202 and the second container 216. The adapter 242 has two openings: one connects to the communication port 220, and the other connects to the second opening 228 of the second container 216, thereby linking the first container 202 and the second container 216. Sealing rings or sealing sleeves are provided between the adapter 242 and the first opening 214, and between the adapter 242 and the second opening 228, to prevent leakage.

Furthermore, the housing 102 includes a first button 204 and a second button 210 located in the area between the first container 202 and the second part 232. The first button 204 is electrically connected to the light-emitting element 162 and the temperature control element 166, and is used to control their activation. The second button 210 is electrically connected to the atomizing core 144 and is used to control its operation. A main control board 158 is provided below the first button 204 and the second button 210, electrically connected to the circuit board 208. When the first button 204 or the second button 210 is pressed, the main control board 158 triggers the corresponding components, emitting electrical signals to open or close the device functions.

Additionally, a battery 160 is provided inside the housing 102, positioned below the first part 224. The battery 160 supplies power to the atomizing core 144, the light-emitting element 162, and the temperature control element 166.

In this embodiment, the positioning of the first button 204, the second button 210, and the battery 160 optimizes the spatial layout between the first container 202 and the second container 216, achieving a compact structure and reducing the overall size of the device.

Referring to FIGS. 12 and 14, in some embodiments, the main body 200 includes a housing 102, a heat-conducting surface 164, and a light-transmitting surface 118. The housing 102 has a third opening 206, which is covered by the heat-conducting surface 164. The heat-conducting surface 164 includes an atomized mist outlet 114 and a light-transmitting hole 250. The light-transmitting surface 118 surrounds the atomized mist outlet 114 and covers the light-transmitting hole 250, thereby forming a light-transmitting surface 118.

The heat-conducting surface 164 is made of a material with high thermal conductivity, such as copper or aluminum, enabling rapid transfer of heat or cold generated by the temperature control element 166 to the skin. The light-transmitting surface 118 can be made of transparent plastic, glass, or ceramic, allowing light to pass through effectively. The housing 102 and the heat-conducting surface 164 can be made of different materials. For example, the housing 102 may be made of plastic, which has lower thermal conductivity than the heat-conducting surface 164, ensuring that the user can comfortably hold the device without the surface becoming too hot or too cold.

Furthermore, the outer surface of the heat-conducting surface 164 includes an annular groove 240 surrounding the atomized mist outlet 114. The light-transmitting surface 118 is embedded into this annular groove 240, with its outer surface flush with the outer surface of the heat-conducting surface 164. Together, the outer surfaces of the light-transmitting surface 118 and the heat-conducting surface 164 form the treatment surface 254. The light-transmitting hole 250 is positioned at the bottom of the annular groove 240.

In some embodiments, the multifunctional therapy device further comprises a dual-liquid atomizing module, wherein the atomizing module includes a first liquid storage chamber and a second liquid storage chamber, each independently connected to an atomizing core. The first and second atomizing cores can operate simultaneously or sequentially to atomize liquids of different compositions, such as water and a nutrient solution, into the atomizing chamber. This allows the device to deliver a combined or customizable treatment to the skin. Optionally, a user-selectable switch or control interface can be provided to select the atomization sequence or combination ratio of the liquids.

In some embodiments, the atomizing module is configured to provide adjustable atomization intensity. The atomization output, including mist particle size and flow rate, can be controlled by varying the operating frequency of the atomizing core or adjusting the speed of the fan disposed within the first housing. This enables the user to select between a gentle, fine mist for sensitive skin or a more intense spray for rapid hydration. Optionally, the main control board can automatically adjust the atomization intensity based on preset modes.

In some embodiments, the multifunctional therapy device further includes at least one sensor configured to monitor skin or environmental conditions. The sensors can include a temperature sensor, a humidity sensor, or a liquid level sensor disposed in the liquid storage chamber. The main control board receives data from the sensors and automatically adjusts the atomization operation, fan speed, phototherapy intensity, or heating/cooling of the treatment surface, thereby optimizing treatment efficiency and safety. Optionally, an alert can be provided to the user if the liquid level is low or if abnormal conditions are detected.

In some embodiments, the phototherapy component comprises multiple light-emitting elements capable of emitting different wavelengths, such as red, blue, green, or infrared light. The user can select a specific wavelength or combination of wavelengths for targeted skin treatment, including hydration, anti-acne, or anti-aging effects. The light-emitting elements can be arranged around the atomizing outlet, allowing atomized mist to simultaneously contact the treated skin. Optionally, the main control board can provide automated cycling between different wavelengths during the treatment session.

In some embodiments, the multifunctional therapy device is configured as a compact, portable, or wearable device. The housing is reduced in size while maintaining the atomizing module, phototherapy component, and heat/cold elements. The device can be powered by a rechargeable battery or replaceable battery cartridge, and may include a detachable liquid container for ease of use and refill. Optionally, the device can include a clip or strap for wearable use, allowing continuous or hands-free operation.

In some embodiments, the device includes an airflow-guiding system that directs atomized mist evenly across the treatment surface. The airflow system can comprise a fan and a set of ducts surrounding the atomizing outlet. This configuration ensures uniform distribution of mist onto the skin and improves the penetration and coverage of hydration. Optionally, the airflow can be adjusted in direction or intensity based on user preference.

The multifunctional therapy device with an atomization module, as described in the above embodiments, has broad industrial applicability. The device can be used in personal care, dermatology, and physiotherapy applications to provide combined skin hydration, phototherapy, and thermal therapy treatments. Its modular and detachable design allows for easy maintenance, cleaning, and replacement of the atomizing module, making it suitable for repeated use in both domestic and professional settings.

The integration of the atomizing module with phototherapy and temperature-control components provides a synergistic effect, enabling simultaneous moisturizing, light-based skin treatment, and heat/cold therapy. This makes the device useful in cosmetic treatment centers, dermatological clinics, physiotherapy practices, and spa facilities.

Furthermore, the compact and ergonomic design of the device allows for user-friendly handling, enabling safe, convenient, and efficient application of treatments. The device is also adaptable for mass production due to its modular construction, standardized components, and simplified assembly process, making it suitable for commercial manufacturing and distribution.

In addition, the device may be further adapted to include alternative atomizing technologies, energy sources, or control mechanisms without departing from the scope of the invention, ensuring versatility across different markets and treatment requirements.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A multifunctional therapy device comprising:
a housing having an atomized mist outlet and a mounting part;
a detachable atomizing module mounted on the mounting part, the atomizing module comprising a liquid storage chamber, an atomizing chamber, and an atomizing core;
a phototherapy component disposed within the housing and corresponding to a light-transmitting surface surrounding the atomized mist outlet;
a heat-conducting surface surrounding the atomized mist outlet; and
a heating element disposed within the heat-conducting surface.

2. The multifunctional therapy device of claim 1, wherein the housing forms a Y-shaped configuration to facilitate user handling and comprising a grip portion, a first extension portion having the atomized mist outlet, and a second extension portion having the mounting part.

3. The multifunctional therapy device of claim 1, wherein the heat-conducting surface and light-transmitting surface are concentric around the atomized mist outlet.

4. The multifunctional therapy device of claim 1, wherein the atomizing module comprises a first conductive element contacting a second conductive element in the housing to power the heating element.

5. The multifunctional therapy device of claim 1, wherein the atomizing module includes an atomizing chamber and a connecting channel allowing liquid from the liquid storage chamber to the atomizing core.

6. The multifunctional therapy device of claim 1, wherein the atomizing module is detachable for cleaning or replacement to reduce maintenance costs.

7. A multifunctional therapy device comprising:
a housing having a mounting part, a mist outlet channel, and an atomized mist outlet;
an atomizing module detachably mounted on the mounting part, the atomizing module comprising a liquid storage chamber, an atomizing chamber, and an atomizing core configured to atomize a liquid from the liquid storage chamber into an atomized mist in the atomizing chamber;
a fan disposed within the housing, the fan is configured to direct the atomized mist toward the atomized mist outlet; and
a main control board electrically connected to the atomizing module.

8. The multifunctional therapy device of claim 7, wherein the atomizing core is a ceramic core with a heating element.

9. The multifunctional therapy device of claim 7, wherein the atomizing core is an ultrasonic atomizing sheet.

10. The multifunctional therapy device of claim 7, wherein the mounting part includes a mounting groove with a first through hole and a second through hole aligned with an air inlet and an air outlet of the atomizing module.

11. The multifunctional therapy device of claim 7, further comprising a sealing pad disposed on the mounting part to prevent leakage of the atomized mist.

12. The multifunctional therapy device of claim 7, further comprising one or more magnetic attractors to secure the atomizing module to the housing.

13. The multifunctional therapy device of claim 7, wherein the atomizing module includes a liquid replenishment port with a detachable sealing cap.

14. A multifunctional therapy device comprising:
a housing;
a liquid storage chamber comprising a first container and a second container, the first container being detachably connected to the housing through a mounting part having a communication port, and the second container having a second opening in fluid communication with a first opening of the first container through the communication port;
the second container further comprising a first part and a second part arranged in an intersecting configuration, the second part including a water outlet and containing absorbent cotton, a portion of the absorbent cotton extending into the first part;
an atomizing core arranged to receive liquid from the water outlet guided by the absorbent cotton and to atomize the liquid; and
an atomized mist outlet configured to discharge an atomized mist generated by the atomizing core,
wherein the second part is inclined such that liquid guided by the absorbent cotton is delivered upward toward the atomizing core in alignment with a spray direction of the atomized mist.

15. The multifunctional therapy device of claim 14, wherein the first container comprises a mounting housing open at both ends and a sealing cap fitted onto an upper end of the mounting housing.

16. The multifunctional therapy device of claim 14, wherein the absorbent cotton is positioned so that one end extends into the first part to draw liquid by capillary action toward the atomizing core.

17. The multifunctional therapy device of claim 14, wherein the atomizing core comprises an atomizing plate disposed within an atomizing chamber located between the water outlet and the atomized mist outlet.

18. The multifunctional therapy device of claim 14, wherein a central axis of the atomized mist outlet is parallel to the central axis of the water outlet to reduce flow resistance and ensure smooth ejection of atomized mist.

19. The multifunctional therapy device of claim 14, wherein the second part gradually tilts away from the first container from bottom to top to facilitate upward liquid delivery through the absorbent cotton.

20. The multifunctional therapy device of claim 14, wherein the liquid storage chamber further comprises an adapter positioned between the first container and the second container, the adapter having two openings respectively connected to the communication port and the second opening and including sealing rings or sealing sleeves to prevent leakage.
